(19) United States
(12) Patent Application Publication
(10) Pub. No.: US 2022/0202420 A1
(43) Pub. Date: Jun. 30, 2022

(54) TISSUE TENSIONING DEVICES, SYSTEMS, AND METHODS

(71) Applicant: Boston Scientific Scimed, Inc., Maple Grove, MN (US)

(72) Inventors: Kathryn Venuto, Maple Grove, MN (US); Christopher R. Deuel, Melrose, MA (US); Ramon Estevez, Lowell, MA (US); Barry Weitzner, Acton, MA (US); Maggie Burds, Peosta, IA (US); Philip Wai Yan Chiu, Kowloon Tong (HK)

(21) Appl. No.: 17/558,081

(22) Filed: Dec. 21, 2021

Related U.S. Application Data
(60) Provisional application No. 63/130,970, filed on Dec. 28, 2020.

(51) Int. Cl. A61B 17/08; A61B 17/10; A61B 17/00; A61B 17/06

(52) U.S. Cl. CPC A61B 17/083; A61B 17/10; A61B 17/00234; A61B 2017/061; A61B 2017/088; A61B 2017/00358; A61B 2017/0034; A61B 17/06066

(57) ABSTRACT

The present disclosure relates to tissue traction devices and systems. In one example, a tissue tensioning system may include a filament having a first end comprising an attachment member, a second end, and a length therebetween extendable externally along a catheter. A first tissue fastener may be engageable with the attachment member and with a target tissue. The second end of the filament may be translatable to adjust a tensional force along the filament.

### PRIORITY

The present application is a non-provisional of and claims the benefit of priority under 35 U.S.C. § 119 to, U.S. Provisional Application Ser. No. 63/130,970, filed Dec. 28, 2020, the disclosure of which is incorporated by reference in its entirety and for all purposes.

### FIELD

The present disclosure relates generally to the field of medical devices. In particular, the present disclosure relates to tissue tensioning devices and systems, e.g., for endoscopic tissue stapling, dissection, resection, or related methods of use thereof.

### BACKGROUND

Accurately and efficiently performing an endoscopic procedure on a layer of target tissue, such as tissue manipulation, stapling, dissection, and/or resection, may include establishing and maintaining tension of the layer of target tissue as the boundaries of the layer of target tissue are manipulated, stapled, dissected, and/or resected. Medical systems may be unable to maintain or adjust tension applied to the layer of target tissue, possibly obstructing a medical professional's view of the layer of target tissue and/or interfering with accessory tools. Medical tools for establishing and maintaining tension on the layer of target tissue may occupy a working channel of an endoscope such that another medical tool cannot be efficiently utilized for a procedure while the layer of target tissue is in tension. These complications may contribute to increased procedure time and complexity.

It is with these considerations in mind that the improvements of the present disclosure may be useful.

### SUMMARY

The present disclosure, in its various aspects, is directed generally to medical devices, and more specifically to tissue tensioning devices and systems, manipulation, stapling, dissection, and/or resection methods, and related delivery systems. Embodiments according to the present disclosure, including as described herein, may decrease complications around tissue removal procedures, such as visualization, procedure time, and procedure complexity. In an aspect described here or otherwise within the scope of the present disclosure, a tissue tensioning system may include a filament having a first end comprising an attachment member, a second end, and a length therebetween extendable externally along a catheter. A first tissue fastener may be engageable with the attachment member and with a target tissue. The second end of the filament may be translatable to adjust a tensional force along the filament.

In various embodiments described herein or otherwise within the scope of the present disclosure, the first tissue fastener may be coupled to the attachment member of the filament. The first tissue fastener and the attachment member of the filament may be extendable within a working channel of the catheter. A second tissue fastener may be engageable with the attachment member and with the target tissue. A system may include a clamping device comprising an anvil and an opposable lever. The clamping device may be configured to clamp about two layers of the target tissue proximate the first tissue fastener. The attachment member may be coupled to the first tissue fastener. The first tissue fastener may be coupled to the target tissue. The attachment member may comprise a loop. The first tissue fastener may comprise opposable jaws. The first tissue fastener may be detachable from within a working channel of the catheter. The second tissue fastener may be coupled to the length of the filament. The second end of the filament may be adjustable to adjust a tensional force along the filament between the first tissue fastener and the second tissue fastener. A system may include a needle comprising a lumen. The filament may be disposable within the lumen with the first tissue fastener coupled to the first end of the filament. The second tissue fastener may be engageable with the second end.

In one aspect described here or otherwise within the scope of the present disclosure, a tissue tensioning system may include a first filament having a first end, a second end, and a length therebetween. A first tissue fastener may be coupled to the first end of the first filament. A second tissue fastener may be coupled to the length of the first filament. The second end of the first filament may be translatable to adjust a tensional force along the first filament between the first tissue fastener and the second fastener.

In various embodiments described herein or otherwise within the scope of the present disclosure, a one-way cleat may be coupled to the first tissue fastener. The second end of the first filament may extend through the one-way cleat such that translating the second end of the first filament away from the second tissue fastener increases tensional force along the first filament between the first tissue fastener and the second tissue fastener. The second end of the first filament may further comprises a knot about the length of the first filament. The knot may be translatable along the length to adjust the tensional force. A grasper may be configured to grasp and translate the second end of the first filament. The first filament, the first tissue fastener, and the second fastener may be extendable through a working channel of a catheter. A system may include a clamping device comprising an anvil and an opposable lever. The clamping device may be configured to clamp about two layers of the target tissue proximate the second tissue fastener. A system may include a second filament having a first end, a second end, and a length therebetween. A third tissue fastener may be coupled to the first end of the second filament. A fourth tissue fastener may be coupled to the length of the second filament. The second end of the second filament may be translatable to adjust a tensional force along the second filament between the third tissue fastener and the fourth tissue fastener.

In one aspect described here or otherwise within the scope of the present disclosure, a tissue tensioning system may include a needle comprising a lumen. A filament may be disposable within the lumen. The filament may comprise a first end comprising an attachment member, a second end, and a length therebetween. A first tissue fastener may be coupled to the second end of the filament. A second tissue fastener may be engageable with the attachment member.

In various embodiments described herein or otherwise within the scope of the present disclosure, the first tissue fastener may be deliverable through a target tissue. The length of the filament may be extendable across a thickness of the target tissue. The second tissue fastener may be engageable with the attachment member and another tissue such that a tensional force along the length of the filament is adjustable. The first tissue fastener may be engageable with a target tissue. The second tissue fastener may be engageable with another tissue substantially opposing the target tissue. The first end of the filament may be coupled to a midportion of the first tissue fastener substantially equidistant between a first end of the first tissue fastener and a second end of the first tissue fastener. The attachment member may comprise a loop. The first tissue fastener may comprise a rod having a delivery configuration wherein a length of the rod may be substantially parallel with the lumen. The rod may have a deployed configuration. The length of the rod may be substantially perpendicular to the lumen. A system may include clamping device comprising an anvil and an opposable lever. The clamping device may be configured to clamp about two layers of the target tissue about the length of the filament. The attachment member may comprise a loop. The first tissue fastener may comprise opposable jaws and may be detachable from within a working channel of a catheter.

### BRIEF DESCRIPTION OF THE DRAWINGS

Non-limiting embodiments of the present disclosure are described by way of example with reference to the accompanying figures, which are schematic and not intended to be drawn to scale. For example, devices may be enlarged so that detail is discernable, but is intended to be scaled down in relation to, e.g., fit within a working channel of a delivery catheter or endoscope. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1A illustrates a tissue tensioning system being oriented toward a layer of target tissue, according to an embodiment of the present disclosure.

FIG. 1B illustrates an exemplary tissue fastener engaging a filament as part of the system of FIG. 1A.

FIG. 1C illustrates exemplary tissue fasteners engaging the filament and the layer of target tissue as part of the system of FIGS. 1A and 1B.

FIG. 1D illustrates an exemplary clamping device clamping the layer of target tissue as part of the system of FIGS. 1A-1C.

FIG. 2A illustrates a tissue tensioning system with tissue fasteners, according to an embodiment of the present disclosure.

FIG. 2B illustrates adjustment of a tensional force along a filament as part of the system of FIG. 2A.

FIG. 2C illustrates an exemplary clamping device clamping the layer of target tissue as part of the system of FIGS. 2A and 2B.

FIG. 3A illustrates a tissue tensioning system being delivered into a layer of target tissue, according to an embodiment of the present disclosure.

FIG. 3B illustrates an exemplary tissue fastener engaging a filament as part of the system of FIG. 3A.

FIG. 3C illustrates an exemplary clamping device clamping the layer of target tissue as part of the system of FIGS. 3A and 3B.

FIG. 4 illustrates a one-way cleat, according to an embodiment of the present disclosure.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g., 1 to 5 includes 1, 1.5, 2, 2.75, 3, 3.80, 4, and 5).

The detailed description should be read with reference to the drawings, which are not necessarily to scale, depict illustrative embodiments, and are not intended to limit the scope of the invention.

A number of medical procedures, including, for example along the digestive and/or biliary tract, utilize medical devices to access tissue intended for manipulation, clamping, dissection, and/or resection (e.g., “target tissue”) within the body. For example, in some current medical procedures (e.g., endoscopic submucosal dissection (ESD), endoscopic mucosal resection (EMR), Peroral Endoscopic Myotomy (POEM), cholecystectomy, Video-Assisted Thoracoscopic Surgery (VATS)), physicians may utilize an endoscope or similar medical device to access and remove diseased lesions. Further, as part of such procedures, a physician may utilize a catheter, such as an endoscope, capable of both accessing the layer of target tissue site while also permitting a dissecting/resecting device or clamping device to be deployed therethrough the catheter to the layer of target tissue. Additionally, in some instances, an endoscope may incorporate features which assist the physician in visualizing and performing the tissue procedure. For example, some endoscopes may include a light and/or camera designed to illuminate and/or visualize the body lumen as the endoscope is navigated and positioned adjacent to the layer of target tissue site. Additionally, some endoscopes may also include a lumen (e.g., a working channel) through which a dissecting/resecting device, grasping member, delivery catheter for the same, or other accessory devices, may be deployed and utilized. Additional visualization methods may be alternatively or additionally employed, e.g., fluoroscopy.

While physicians are becoming more proficient at removing diseased lesions from within the body (e.g., within the digestive tract, abdominal cavity, thoracic cavity, etc.), present tissue tensioning methods may continue to be inefficient to the physician. For example, in some instances, poor visualization and poor ability to engage and manipulate tissue may result in a prolonged procedure. An aspect of EMR/ESD that may be difficult is the positioning and maneuvering (e.g., tensioning) of the layer of target tissue. In some EMR/ESD procedures, physicians may use separate devices to each provide traction to tissue. Such procedures may include multiple device exchanges and extended procedure times. Such systems may be unable to maintain or adjust traction, or tension, applied to the layer of target tissue, and/or may maintain or adjust tension applied to the layer of target tissue in an inefficient or inconsistent manner.

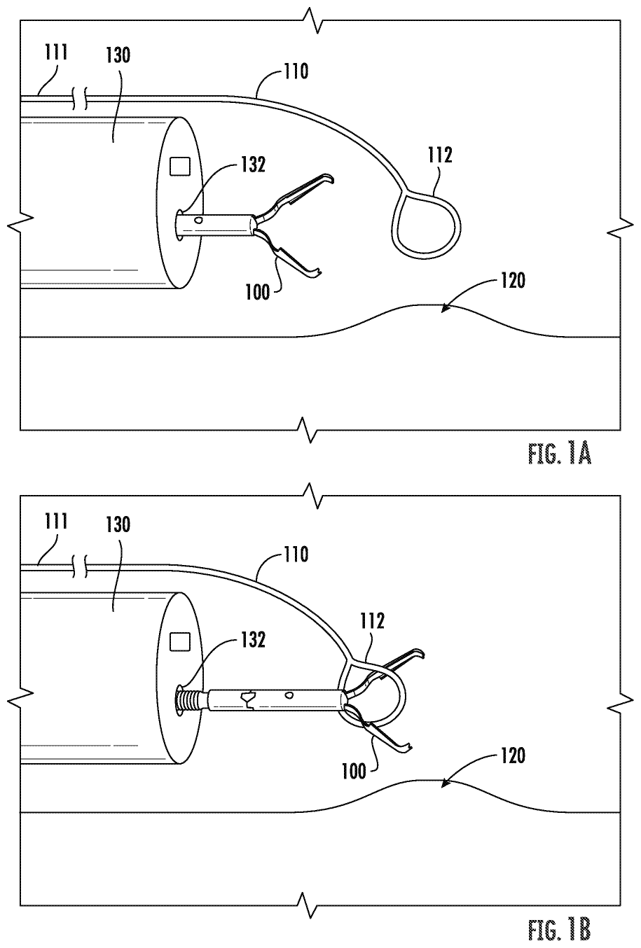

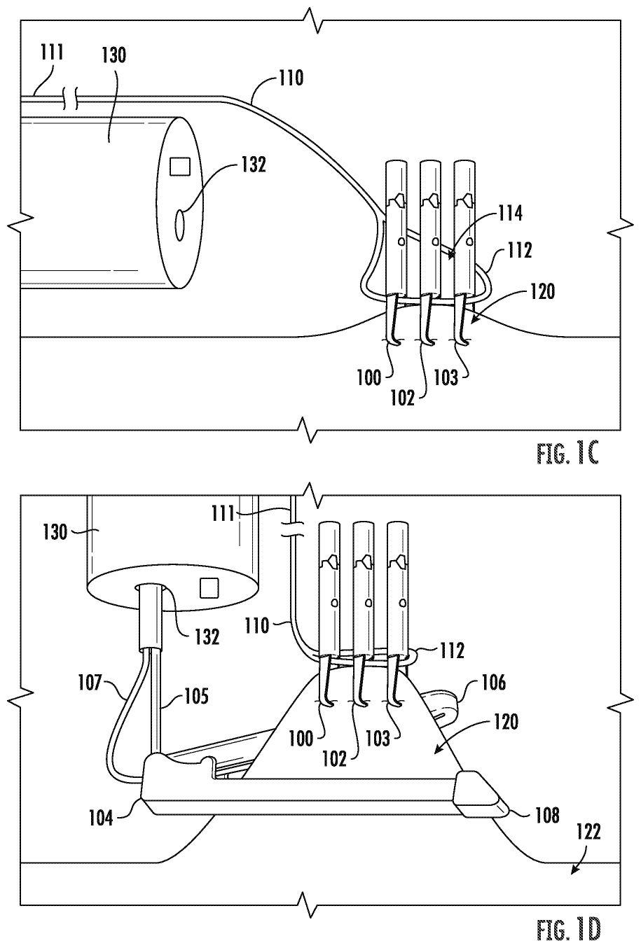

Referring to FIG. 1A, an embodiment of a tissue tensioning system is illustrated as being oriented toward a layer of target tissue 120 to be placed under tension for a procedure as described throughout this disclosure. A catheter 130 (e.g., an endoscope) having a working channel 132 allows for visualization of the layer of target tissue 120 and for delivery and visualization of one or more devices of the system. A filament 110 is being delivered toward the layer of target tissue 120. The filament 110 includes a first end having an attachment member 112 and a second end 111 extending proximally along the catheter 130 toward the medical professional for manipulation. The attachment member 112 may include an engagement feature for another medical device to engage, e.g., the attachment member 112 may be an open loop (e.g., a snare) or another atraumatic shape engageable by a medical instrument. In various embodiments described herein, a filament may be flexible along its entire length or may have an end portion that is more rigid than a remainder of the filament. A first tissue fastener 100 extending from the working channel 132 is being delivered toward the layer of target tissue 120 and the attachment member 112.

Referring to FIG. 1B, the tissue tensioning system of FIG. 1A is illustrated with the first tissue fastener 100 engaging the attachment member 112. The tissue fastener 100 is in an open position with its jaws able to accept the attachment member 112 such that the attachment member extends between the jaws. With the tissue fastener 100 engaging the attachment member 112, movement of the tissue fastener 100 also moves the filament 110 via the attachment member 112. Alternatively, the attachment member 112 may be preloaded as engaged with the tissue fastener 100 (e.g., with the tissue fastener 100 engaging the attachment member 112 within the working channel 132). If the attachment member 112 is preloaded as engaged with the tissue fastener 100, the tissue fastener 100 does not need to be manipulated to engage the attachment member 112 within the patient during the procedure. Together, the tissue fastener 100 and the attachment member 112 may be manipulated toward the layer of target tissue 120. The open jaws of the first tissue fastener 100 engaged with the attachment member 112 may be manipulated to also engage the layer of target tissue 120 between the jaws.

Referring to FIG. 1C, the tissue tensioning system of FIGS. 1A and 1B is illustrated with the first tissue fastener 100 engaging the attachment member 112 and the layer of target tissue 120. The filament 110 and layer of target tissue 120 are coupled together via the attachment member 112 and the layer of target tissue 120 being engaged by the jaws of the first tissue fastener 100. In a substantially similar process, one or more additional tissue fasteners (e.g., a second tissue fastener 102 and a third tissue fastener 103) are delivered through the working channel 132 of the catheter 130. The additional tissue fastener(s) 102, 103 are engaging the attachment member 112 and the layer of target tissue 120. The attachment member 112 may have an open aperture 114 large enough to accommodate further tissue fasteners, including the first and second fasteners 102, 103 or more. The additional tissue fasteners 102, 103 engage the attachment member 112 and the layer of target tissue 120, while the first tissue fastener 100 is already engaged with the attachment member 112 and the layer of target tissue 120, and with minimal manipulation of the tissue fasteners 102, 103 because the engagement member 112 is open to accepting multiple tissue fasteners. The filament 110 is coupled to the layer of target tissue 120 at multiple locations via the tissue fasteners 100, 102, 103. Each tissue fastener 100, 102, 103 may engage the target tissue 120 such that as much of the target tissue 120 as possible is engaged by each tissue fastener 100, 102, 103 while minimizing slippage (i.e., partial or complete disengagement of the tissue fasteners 100, 102, 103 from the target tissue 120 after engagement). Although three tissue fasteners 100, 102, 103 are illustrated, in various embodiments any number of tissue fasteners may be used, e.g., 1, 2, 4, 5, 6, 8, 10, 15, 20, 50, 100, etc.

Referring to FIG. 1D, the tissue tensioning system of FIGS. 1A through 1C is illustrated with the tissue fasteners 100, 102, 103 engaging the attachment member 112 and the layer of target tissue 120. The catheter 130 and the length of the filament 110 are oriented such that they are substantially perpendicular to the layer of target tissue 120. Because the filament 110 is coupled to the layer of target tissue 120 via the tissue fasteners 100, 102, 103, the second end 111 of the filament 110 can be translated proximally (i.e., substantially normal to the layer of target tissue 120) thereby applying tensile force to the layer of target tissue 120 via the tissue fasteners 100, 102, 103. The tensile force pulls the layer of target tissue 120 away from the surrounding tissue 122. The tissue fasteners 100, 102, 103 translate the tensile force across the layer of target tissue 120 to portions engaged with the tissue fasteners 100, 102, 103. The tensile force may be applied to multiple portions of the layer of target tissue 120 to direct the size and shape of the lifted layer of target tissue 120. The tissue fasteners 100, 102, 103 may be more loosely or more tightly clustered along the layer of target tissue 120 to spread out or narrow the surface area of the layer of target tissue 120 affected by the tensile force. The tensional force of the filament created by the tensile force applied to the second end 111, along the filament 110, and to the layer of target tissue 120, may be adjusted by proximally or distally translating the second end 111 of the filament 110 and/or adjusting the force applied to the second end 111 of the filament 110. Adjusting the tensional force of the filament 110 applied to the layer of target tissue 120 may also adjust the amount and the distance the layer of target tissue 120 is lifted away from the surrounding tissue 122. With the filament 110 extending generally along the catheter 130 and applying the tensional force to the layer of target tissue 120, the working channel 132 of the catheter is free to deliver a medical device for manipulating the tensioned layer of target tissue 120. In FIG. 1D, an exemplary medical device for manipulating the tensioned layer of target tissue 120 is a clamping device 104 extending from the working channel 132 toward the layer of target tissue 120. The clamping device 104 includes an opposable lever 108 that can be manipulated away from and towards an anvil 106. The lever 108 and anvil 106 are extended about the tensioned layer of target tissue 120. The clamping device 104 is extendable along axial support 105. The orientation of the lever 108 and anvil 106 are manipulated with respect to the axial support 105 by manipulation of a manipulating member 107. The manipulating member 107 of the clamping device 104 may be manipulated such that the lever 108 and anvil 106 can extend about the layer of target tissue 120 at an angle substantially normal to the tensional force from the filament 110. Although a clamping device is illustrated, in various embodiments described herein, other medical devices (e.g., a stapler, a knife, a cutting tool, or the like) may instead or additionally be used to engage the layer of target tissue 120.

In various embodiments described herein, a medical device may be used to engage tensioned layer of target tissue. For example, a clamping device may include an opposable lever and an anvil, a pair of opposable levers, a pair of opposable jaws, or the like. A clamping device may hold, cut, staple, resect, dissect, and/or sever tensioned layer of target tissue. In various embodiments, a full layer of tensioned layer of target tissue may be engaged for full-thickness resection by a clamping device surrounding the tensioned layer of target tissue such that two thicknesses of the layer of target tissue are engaged between the clamping device. The clamping device may cut across one or more layers of the tissue and/or apply staples into and/or across one or more layers of the tissue. Full-thickness resection may advantageously treat target tissue without undesirable tearing or perforation of the layer of target tissue compared to other tissue engaging techniques such as cutting.

In various embodiments described herein, a filament may extend external to the catheter during delivery and/or deployment. The filament may be freely extending along the catheter or may be coupled along the length of the catheter. Alternatively, the filament may extend partially or completely within a working channel, a second channel of the catheter, an external channel of the catheter, and/or through a clip, pin, or pulley.

In various embodiments described herein, a tissue other than the target tissue, e.g., substantially opposing the target tissue, may be engaged to apply tension between the target tissue and the other tissue along a filament therebetween. One or more of these tissues may be within a GI lumen where e.g., a diseased target tissue along the GI lumen that is lifted towards a substantially opposing side of the same GI lumen to the other tissue that may be continuous with the target tissue (e.g., along the tissue wall of the GI lumen).

In various embodiments herein, a filament may be illustrated with a particular number of attachment members, however, any number of attachment members may be used. Additionally, the one or more attachment members may be located at one or more ends of a filament and/or at one or more locations along the length of the filament, e.g., to provide additional locations for engagement by a tissue fastener.

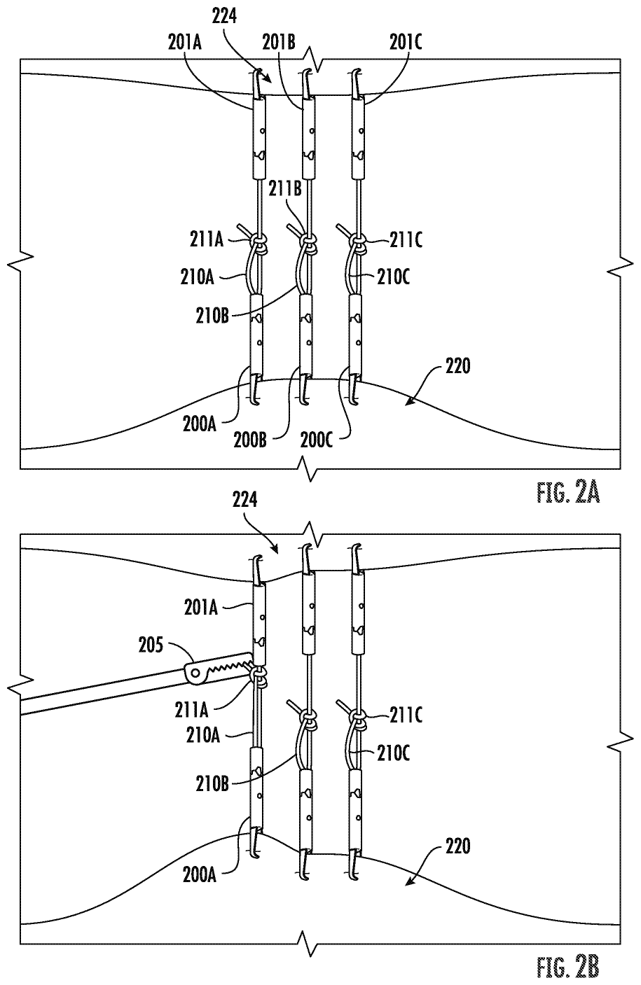

Referring to FIG. 2A, an embodiment of a tissue tensioning system according to the present disclosure is illustrated including first tissue fasteners 200A, 200B, 200C engaging a layer of target tissue 220. The first tissue fasteners 200A, 200B, 200C are each coupled to a length of a corresponding filament 210A, 210B, 210C. Second tissue fasteners 201A, 201B, 201C are coupled to an end of each corresponding filament 210A, 210B, 210C. Each of the first tissue fasteners 200A, 200B, 200C are engaged with the layer of target tissue 220 and each of the second tissue fasteners 201A, 201B, 201C are engaged with another tissue 224. Each of the filaments 210A, 210B, 210C extending from the first tissue fasteners 200A, 200B, 200C to the second tissue fasteners 201A, 201B, 201C couple the layer of target tissue 220 to the other tissue 224 (i.e., from each of the first tissue fasteners 200A, 200B, 200C along each of the filaments 210A, 210B, 210C to each of the second tissue fasteners 201A, 201B, 201C). A tensional force along each of the filaments 210A, 210B, 210C and between the layer of target tissue 220 and the other tissue 224 may be selected, e.g., by firstly engaging a first tissue fastener 200A, 200B, 200C to the layer of target tissue 220 and engaging a corresponding second tissue fastener 201A, 201B, 201C to a location on the other tissue 224 and/or other anatomy. A location selected along the other tissue 224 for the second tissue fastener 201A, 201B, 201C that is farther from the layer of target tissue 220 will apply a larger tensional force to the corresponding filament 210A, 210B, 210C than a second tissue fastener 201A, 201B, 201C location along the other tissue 224 that is closer to the layer of target tissue 220. Although three pairs of first and second tissue fasteners 200A, 200B, 200C, 201A, 201B, 201C are illustrated, any number of tissue fasteners 200A, 200B, 200C, 201A, 201B, 201C may be used, e.g., 1, 2, 4, 5, 6, 8, 10, 15, 20, 50, etc. The tissue fasteners 200A, 200B, 200C, 201A, 201B, 201C may be more loosely or more tightly clustered along the layer of target tissue 220 and/or other tissue 224 to spread out or narrow in the surface area of the layer of target tissue 220 affected by the tensional force(s). Selecting a desirable tensional force applied to the layer of target tissue 220 may additionally or alternatively be adjusted by manipulating one or more filaments 210A, 210B, 210C. An end portion 211A, 211B, 211C of each filament 210A, 210B, 210C is movable along the length of each filament 210A, 210B, 210C to adjust the tensional force between the fasteners 200A, 200B, 200C, 201A, 201B, 201C (and thereby adjusting the tensional force between the tissues 220, 224). For example, FIG. 2A illustrates each end portion 211A, 211B, 211C of the filaments 210A, 210B, 210C having a taut-line hitch knot that may be moved along the length of its filament 210A, 210B, 210C.

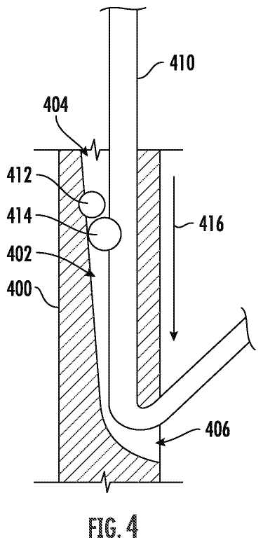

Referring to FIG. 2B, the embodiment of a tissue tensioning system of FIG. 2A is illustrated with the tensional force between the first tissue fastener 200A and the second tissue fastener 201A being adjusted. A medical device 205 (e.g., a grasper with opposable jaws is illustrated, but another manipulation device may instead be used) is moving the end portion 211A of the filament 210A away from the first tissue fastener 200A such that a portion of the length of the filament 210A between the first and second tissue fasteners 200A, 201A moves through the first tissue fastener 200A, bringing the first and second tissue fasteners 200A, 201A closer to each other. Because the first tissue fastener 200A is engaged with the layer of target tissue 220 and the second tissue fastener 201A is engaged with the other tissue 224, adjusting the end portion 211A of the filament 210A will adjust the tensional force and/or distance between the tissues 220, 224. Although FIGS. 2A-2C illustrate taut-line hitch knots slidable along the length of the filaments 210A, 210B, 210C, in various embodiments alternative length and/or tensional force adjusting features may be used along the filament(s) and/or the fastener(s) (e.g., other knots, one-way cleats (e.g., as illustrated in FIG. 4), brakes, clamps, cinches, one-way sockets, or the like). The end portions 211A, 211B, 211C of the filaments 210A, 210B, 210C may be adjusted to have varying or similar tensional forces between the first tissue fasteners 200A, 200B, 200C and the second tissue fasteners 201A, 201B, 201C to achieve a desirable tensional force profile across the layer of target tissue 220.

Referring to FIG. 2C, the embodiment of the tissue tensioning system of FIGS. 2A and 2B is illustrated with the three end portions 211A, 211B, 211C adjusted such that the three filaments 210A, 210B, 210C between the first tissue fasteners 200A, 200B, 200C and the second tissue fasteners 201A, 201B, 201C have a tensional force there-along that is larger than that of arrangements in FIG. 2A and the layer of target tissue 220 is lifted away from surrounding tissue 222. Adjusting the length(s) and/or tensional force(s) of the filament(s) 210A, 210B, 210C may adjust the amount and the distance the layer of target tissue 220 is lifted away from the surrounding tissue 222. With the layer of target tissue 220 lifted from the surrounding tissue 222, a medical device for manipulating the tensioned layer of target tissue 220 may be introduced to the layer of target tissue 220. In FIG. 2C, an exemplary medical device for manipulating the tensioned layer of target tissue 220 is a clamping device 204. The clamping device 204 includes an opposable lever 208 that can be manipulated away from and towards an anvil 206. The lever 208 and anvil 206 are extended about the tensioned layer of target tissue 220. Although a clamping device is illustrated, in various embodiments described herein other medical devices (e.g., a stapler, a knife, a cutting tool, or the like) may instead or additionally be used to engage the layer of target tissue 220.

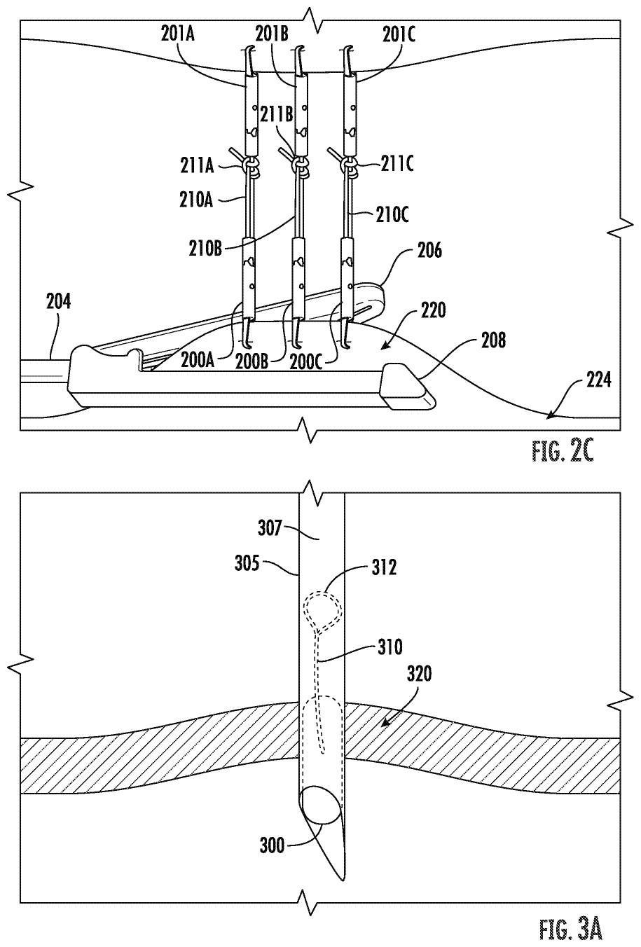

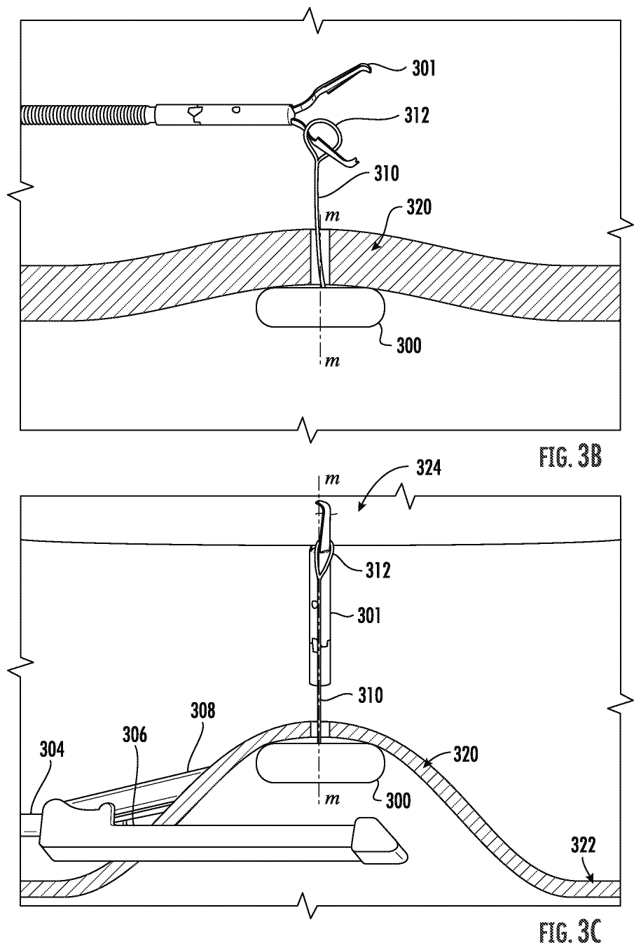

Referring to FIG. 3A, an embodiment of a tissue tensioning system being delivered into a layer of target tissue 320, according to an embodiment of the present disclosure, is illustrated. A needle 305, e.g., a hollow needle, a hypotube, a hypodermic needle, or the like, is inserted into the layer of target tissue 320. In various embodiments, the needle 305 may be inserted partially into or across one or more layers or a thickness of a layer of the layer of target tissue 320. A filament 310 is disposed within a lumen 307 of the needle 305. The filament 310 has a first tissue fastener 300 coupled to a first end of the filament 310 (e.g., coupled by overmolding, tying, knotting, looping, adhering, a combination thereof, or the like). The first tissue fastener 300 is oriented within the lumen 307 such that a length of the first tissue fastener is substantially parallel with the lumen 307. An outer diameter of the first tissue fastener 300 substantially matches a diameter of the lumen 307 (i.e., an inner diameter of the needle 305) such that a cross-section of the lumen 307 is substantially filled by the first tissue fastener 300. Because the first tissue fastener 300 substantially fills the lumen 307, the layer of target tissue 320 is not substantially cored by the needle 305 (e.g., compared to a vacant lumen 307 when the needle 305 is delivered within the layer of target tissue 320). The first tissue fastener 300 includes filleted or dulled edges reducing friction between the first tissue fastener 300 within the lumen 307 against walls of the needle 305 and/or reducing friction with anatomy (i.e., compared to non-filleted or non-dulled edges). The filament 310 includes an attachment member 312 at a second end of the filament 310. The first tissue fastener 300 coupled to the filament 310 may be deployed from the lumen 307 by, e.g., a fluid pressure applied within the lumen 307 proximal of the first tissue fastener 300, a rod pushing proximal of the first tissue fastener 300, or the like. The needle 305 is delivered into and extending across the layer of target tissue 320. The first tissue fastener 300 is positioned at least partially across the layer of target tissue 320 while the attachment member 312 of the filament 310 is at an opposing side of the layer of target tissue 320 (e.g., the first tissue fastener 300 may be delivered subcutaneously within or across the layer of target tissue 320 wall with the attachment member 312 proximally above the layer of target tissue 320). From this position, the first tissue fastener 300 may be deployed out of the lumen 307 on one side of the layer of target tissue 320 and the attachment member 312 may be deployed from the lumen 307 by withdrawing the needle 305 from the layer of target tissue 320 and the filament 310.

Referring to FIG. 3B, the tissue tensioning system of FIG. 3A is illustrated with the first tissue fastener 300 deployed at one side of the layer of target tissue 320, the filament 310 extending across the layer of target tissue 320, and the attachment member 312 deployed at another side of the layer of target tissue 320. A second tissue fastener 301 is engaging the deployed attachment member 312. Although the configuration of the first tissue fastener 300 illustrated in FIG. 3B may occur upon delivery, the second tissue fastener 301 may be used to manipulate the attachment member 312, e.g., away from the layer of target tissue 320, such that the filament 310 is pulled along with the first tissue fastener 300. Because the first end of the filament 310 is coupled substantially at a midportion of the first tissue fastener 300 (i.e., a portion along the length of the first tissue fastener 300 that is substantially equidistant to both ends of the first tissue fastener 300), pulling the filament 310 away from the first tissue fastener 300 will orient the length of the first tissue fastener 300 such that it is substantially normal to a mid-axis m. Mid-axis m extends substantially along the pulled filament 310 across the tissue 320 and substantially normal to the length of the first tissue fastener 300 such that the length of the first tissue fastener 300 extends substantially parallel with the layer of target tissue 320. In this configuration, the first tissue fastener 300 provides resistance against the layer of target tissue 320 as the attachment member 312 is pulled away from the layer of target tissue 320.

Referring to FIG. 3C, the tissue tensioning system of FIGS. 3A and 3B is illustrated with the second tissue fastener 301 engaged with another tissue 324. The second tissue fastener 301 was used to manipulate the attachment member 312 away from the layer of target tissue 320, causing the filament 310 to pull the first tissue fastener 300 against the layer of target tissue 320 with greater tensional force than that of FIG. 3B. This increase in tensional force substantially along the mid-axis m extending from the middle of the first tissue fastener 300, through the filament 310, and to an engagement point of the second tissue fastener 301 along the other tissue 324 lifts the layer of target tissue 320 away from the surrounding tissue 322. With the layer of target tissue 320 tensioned and lifted away from the surrounding tissue 322, a medical device for manipulating the tensioned layer of target tissue 320 may be introduced to the layer of target tissue. In FIG. 3C, an exemplary medical device for manipulating the tensioned layer of target tissue 320 is a clamping device 304. The clamping device 304 includes an opposable lever 308 that can be manipulated away from and towards an anvil 306. The lever 308 and anvil 306 are extended about the tensioned layer of target tissue 320. Although a clamping device is illustrated, in various embodiments described herein other medical devices (e.g., a stapler, a knife, a cutting tool, or the like) may instead or additionally be used to engage the layer of target tissue 320. The amount of tensional force along the filament 310 applied to the layer of target tissue 320 may be adjusted, e.g., by positioning or repositioning the second tissue fastener 301 to a location along the other tissue 324 that is closer to, farther away from, or at an alternative angle with respect to the layer of target tissue 320. Although one filament 310 coupled to a first tissue fastener 300 and engaged by a second tissue fastener 301 is illustrated, additional filaments and/or tissue fasteners may be used, e.g., to alter the amount of tensional force applied to or the lifted shape of the layer of target tissue 320. In various embodiments, one or more additional tissue fasteners may further engage the filament 310 to manipulate and/or adjust tensional force.

In various embodiments described herein, a tissue fastener may additionally or alternatively include one or more features for adjusting a tensional force along a filament. For example, with reference to FIG. 4, a one-way cleat is illustrated, according to an embodiment of the present disclosure. In the illustrated embodiment a cross-section of a portion of a tissue fastener 400 is illustrated with a channel 402 having an inlet 404 and an outlet 406. A filament 410 described herein extends through the channel 402 and may be translated in the general direction 416 to increase tensional force in the filament 410. The filament 410 may be locked into a position to maintain a tensional force and released by a stop member 412 that is fixed and a roller 414 that is movably positioned in an angled portion of the channel 402. The stop member 412 and the roller 414 are illustrated as being cylindrical in shape, though the shape is not critical and other shapes are contemplated. The roller 414 freely rolls along and in contact with the filament 410 as the filament 410 tangentially moves along the roller 414 within the channel 402 towards the outlet 406. The stop member 412 substantially prevents the roller 414 from rolling along with tangential movement of the filament 410 in a direction substantially opposing the direction 416.

In various embodiments, a tissue fastener may be rotatable or may rotate about a filament. A tissue fastener may be repositionable before, during, and/or after a procedure. A tissue fastener may be a single use tissue fastener (i.e., not repositionable). A medical procedure such as resecting of a tissue may be performed with a clamping device engaging one or more tissues in tension. During and/or after the procedure, tension may be released by severing a portion of the device, such as a filament, and/or an attachment member.

In various embodiments, a filament or features thereof may comprise various shapes such as a loop, s snare, a hook, an anchor, a knot, a barb, an eyelet, a combination thereof, or the like. In various embodiments, a filament may comprise a polymer strand (e.g., polypropylene, polyester, nylon, polyethylene, elastic polymers including thermoplastic elastomer (TPE), polyisoprene, silicone, and/or the like), a metal wire (e.g., stainless steel, titanium, cobalt-chrome, nitinol, and/or the like), and/or a natural fiber (e.g., cotton, wool, silk, and/or the like). A filament may have a material strength configured to fail at a pre-determined load as a safety feature to limit an amount of tension in the traction band and the surrounding tissue. One or more filaments may be visually marked such that the filaments are visually distinguishable with respect to other filaments. For example, the filaments may vary in colors, patterns, or radiopacity such that a medical professional can easily identify a selected filament meant for fixation to a layer of target tissue, to an anchoring tissue, to a second anchoring tissue, for releasing from a connector body, etc.

In various embodiments, some steps of assembling a tissue tensioning device or system may occur outside of the patient's body, while other steps involved in assembling the tissue tensioning device may occur within the patient. The steps described herein do not necessarily occur in a specific order and/or timing.

The medical devices used with various embodiments of the devices, systems, and methods herein are not limited to those illustrated and discussed but may include a variety of medical instruments (e.g., ablative elements, biopsy needles, injection needles, scissors, graspers, clips, clamps, staplers, knives, etc.).

In various embodiments, an access area beneath and about a layer of target tissue may be visualized. Visualization may be optical, fluoroscopic, ultrasonic, etc. The visualization of the area beneath and about the layer of target tissue may not be adequately revealed for the medical professional to manipulate a medical device to or near the layer of target tissue. The medical professional may deliver and deploy a tissue tensioning device or system to the layer of target tissue and another tissue at a length and/or at a tension that reveals the access area for the procedure. The medical professional may adjust the length or tension of the system based on visualization of the layer of target tissue or nearby area.

In various embodiments, a filament may be engaged with a variety of different fasteners configured to engage a filament along with a tissue, such as a clip, an anchor, a screw, a pin, or the like. For example, a fastener contemplated for use with a tissue tensioning device may include a biased-open configuration configured to move to a closed/clamped configuration upon actuation by a handle assembly. In addition, or alternatively, a tissue fastener contemplated for use with a disclosed tissue tensioning device or system may include a biased-closed configuration configured to move an open configuration upon actuation of a distal end effector (e.g., squeezing) by a proximal handle assembly. In addition, or alternatively, fasteners other than detachable/releasable tissue fasteners may be used to secure/engage the attachment members of the disclosed tissue tensioning device to the wall of a body lumen, such as non-repositionable fasteners.

In various embodiments described herein, a method of applying tension to a tissue may include delivering a filament toward a layer of target tissue. A tissue fastener may engage an attachment member of the filament and the layer of target tissue. Additional tissue fastener(s) may engage the attachment member and the layer of target tissue. The filament may be moved, adjusting a tensional force along the filament and applied to the layer of target tissue. A clamping device may be delivered about the layer of target tissue and clamp the layer of target tissue.

In various embodiments described herein, a method of applying tension to a tissue may include delivering a first tissue fastener coupled to a filament to engage a layer of target tissue. A second tissue fastener coupled to the filament may be delivered to engage another tissue. An end portion of the filament may be adjusted to adjust a tensional force between the first tissue fastener and the second tissue fastener, thereby adjusting a tensional force applied to the layer of target tissue. A clamping device may be delivered about the layer of target tissue and clamp the layer of target tissue.

In various embodiments described herein, a method of applying tension to a tissue may include inserting a needle into a layer of target tissue, the needle having a lumen containing a filament coupled to a tissue fastener. The tissue fastener may be deployed into the layer of target tissue, across one or more layers, and/or proximate a side of the layer of target tissue. An attachment member of the filament may be deployed at an opposing side of the layer of target tissue. A tissue fastener may be delivered to engage the attachment member and another layer of target tissue, thereby applying a tensional force along the filament and to the layer of target tissue. A clamping device may be delivered about the layer of target tissue and clamp the layer of target tissue.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A tissue tensioning system, comprising:
a filament having a first end comprising an attachment member, a second end, and a length therebetween extendable externally along a catheter; and
a first tissue fastener engageable with the attachment member and with a target tissue;
wherein the second end of the filament is translatable to adjust a tensional force along the filament.

2. The tissue tensioning system of claim 1, wherein the first tissue fastener is coupled to the attachment member of the filament, and wherein the first tissue fastener and the attachment member of the filament are extendable within a working channel of the catheter.

3. The tissue tensioning system of claim 1, further comprising a second tissue fastener engageable with the attachment member and with the target tissue.

4. The tissue tensioning system of claim 1, further comprising a clamping device configured to clamp about two layers of the target tissue proximate the first tissue fastener.

5. The tissue tensioning system of claim 1, wherein the attachment member is coupled to the first tissue fastener and the first tissue fastener is coupled to the target tissue.

6. The tissue tensioning system of claim 1, wherein the attachment member comprises a loop and the first tissue fastener comprises opposable jaws.

7. The tissue tensioning system of claim 1, further comprising the second tissue fastener coupled to the length of the filament, wherein the second end of the filament is adjustable to adjust a tensional force along the filament between the first tissue fastener and the second tissue fastener.

8. The tissue tensioning system of claim 1, further comprising a needle comprising a lumen, and wherein the filament is disposable within the lumen with the first tissue fastener coupled to the first end of the filament, and wherein the second tissue fastener is engageable with the second end.

9. A tissue tensioning system, comprising:
a first filament having a first end, a second end, and a length therebetween;
a first tissue fastener coupled to the first end of the first filament; and
a second tissue fastener coupled to the length of the first filament, wherein the second end of the first filament is translatable to adjust a tensional force along the first filament between the first tissue fastener and the second fastener.

10. The tissue tensioning system of claim 9, further comprising a one-way cleat coupled to the first tissue fastener wherein the second end of the first filament extends through the one-way cleat such that translating the second end of the first filament away from the second tissue fastener increases tensional force along the first filament between the first tissue fastener and the second tissue fastener.

11. The tissue tensioning system of claim 9, wherein the second end of the first filament further comprises knot about the length of the first filament, the knot translatable along the length to adjust the tensional force.

12. The tissue tensioning system of claim 9, further comprising a grasper configured to grasp and translate the second end of the first filament.

13. The tissue tensioning system of claim 9, wherein the first filament, the first tissue fastener, and the second fastener are extendable through a working channel of a catheter.

14. The tissue tensioning system of claim 9, further comprising a clamping device configured to clamp about two layers of the target tissue proximate the second tissue fastener.

15. The tissue tensioning system of claim 9, further comprising:
a second filament having a first end, a second end, and a length therebetween;
a third tissue fastener coupled to the first end of the second filament; and
a fourth tissue fastener coupled to the length of the second filament, wherein the second end of the second filament is translatable to adjust a tensional force along the second filament between the third tissue fastener and the fourth tissue fastener.

16. A tissue tensioning system, comprising:
a needle comprising a lumen;
a filament disposable within the lumen, the filament comprising a first end comprising an attachment member, a second end, and a length therebetween;
a first tissue fastener coupled to the second end of the filament; and
a second tissue fastener engageable with the attachment member.

17. The tissue tensioning system of claim 16, wherein the first tissue fastener is deliverable through a target tissue, the length of the filament is extendable across a thickness of the target tissue, and the second tissue fastener is engageable with the attachment member and another tissue such that a tensional force along the length of the filament is adjustable.

18. The tissue tensioning system of claim 16, wherein the first tissue fastener is engageable with a target tissue and the second tissue fastener is engageable with another tissue substantially opposing the target tissue.

19. The tissue tensioning system of claim 16, wherein the first end of the filament is coupled to a midportion of the first tissue fastener substantially equidistant between a first end of the first tissue fastener and a second end of the first tissue fastener, and wherein the attachment member comprises a loop.

20. The tissue tensioning system of claim 16, wherein the first tissue fastener comprises a rod having a delivery configuration wherein a length of the rod is substantially parallel with the lumen and wherein the rod has a deployed configuration wherein the length of the rod is substantially perpendicular to the lumen.
